(19) United States
(12) Patent Application Publication
(10) Pub. No.: US 2005/0196416 A1
(43) Pub. Date: Sep. 8, 2005

(54) Dispersions prepared by use of self-stabilizing agents

(76) Inventors: James E. Kipp, Wauconda, IL (US); Mark J. Doty, Grayslake, IL (US); Christine L. Rebbeck, Algonquin, IL (US)

Correspondence Address: Baxter Healthcare Corporation, One Baxter Parkway - DF2-2E, Deerfield, IL, 60015, US

(21) Appl. No.: 11/043,314

(22) Filed: Jan. 26, 2005

Related U.S. Application Data
(60) Provisional application No. 60/542,372, filed on Feb. 5, 2004.

(51) Int. Cl. A61K 38/17; A61K 48/00; A61K 9/127; A61K 31/557

(52) U.S. Cl. 424/400; 514/12; 514/44; 514/573

(57) ABSTRACT

The present invention relates to a dispersion of an active agent, which includes a multiphase system of an organic phase and an aqueous phase. The agent, preferably poorly water soluble, possesses surface active properties and itself serves as a dispersant or a stabilizer for the dispersion. The dispersion is suitable for pharmaceutical, veterinary, cosmetic, and agricultural applications, and is suitable for in vivo delivery, particularly by parenteral routes.

### CROSS-REFERENCE TO RELATED APPLICATIONS

The present invention claims priority to U.S. Provisional Patent Application No. 60/542,372

### BACKGROUND OF THE INVENTION

1. Technical Field

The present invention relates to a dispersion of an organic compound, which includes a multiphase system of an organic phase and an aqueous phase. The agent, preferably poorly water soluble, possesses surface-active properties and serves as a dispersant or stabilizer in the dispersion. The dispersion may be an emulsion, suspension, or association colloid (micellar dispersion) and is suitable for pharmaceutical, veterinary, cosmetic and agricultural applications.

2. Background Art

Water-insoluble organic materials, solid or liquid, provide challenges in formulation as stable, homogeneously dispersed multiphase systems. Preparations of this nature have important applications in efficacious delivery of the active ingredient. In particular, substances that are insoluble in water can have significant benefits when formulated as stable dispersions (e.g., suspensions, emulsions, or micellar dispersions) of submicron particles. Control of particle size and long-term stability are essential for safe and efficacious use of these formulations.

In pharmaceutical applications, particularly in parenteral drug delivery, particles must be less than seven microns in diameter to safely pass through capillaries without causing emboli (Allen et al., 1987; Davis and Taube, 1978; Schroeder et al., 1978; Yokel et al., 1981). One solution to this problem is the production of extremely small particles of the insoluble drug candidate and the creation of a microparticulate or nanoparticulate suspension. In this way, drugs that were previously unable to be formulated in an aqueous based system can be made suitable for parenteral administration. Suitability for parenteral administration includes small particle size (<7 μm), low toxicity (as from toxic formulation components or residual solvents), and bioavailability of the drug particles after administration.

The parenteral administration of such poorly water soluble pharmaceutical agents has been achieved in the past using emulsions composed of a hydrophobic solvent (e.g., oil) and a stabilized drug dispersed within an aqueous medium, such as a buffer solution or normal saline solution. Emulsions have been used to deliver poorly water-soluble drugs such as fat-soluble vitamins (e.g., vitamins A, D and E), and hydrophobic pharmaceuticals such as propofol. Particle size ranges between 100 and 700 nm. Many emulsions may be heat sterilized, and many can be designed with small particle size (less than 200 nm) appropriate for sterile filtration. The pharmacodynamics of a drug delivery system may be tailored by altering the size distribution and coating of the oil droplets. In this manner, passive targeting may be directed at sites of disease. Typically, emulsions require the application of high shear mixing to break down the oil droplets to a desired size. Low temperature processes may be designed to accommodate heat-sensitive compounds.

However, the preparation of emulsions typically requires the use of emulsifying agents such as phospholipids (e.g., lecithin), fatty acids, long-chain alcohols or bile salts. The emulsifier coats each oil droplet and at least one ionic component provides a charged layer (Stem Layer) near the droplet surface. The positive electrostatic potential barrier created by this charge separation prevents coacervation of the droplets. In many instances, because of high-dose requirements for some pharmaceuticals, surface-active excipients must be used at a high concentration that is sufficient to emulsify the oil needed to accommodate the drug. It is advantageous to minimize or even eliminate many of the emulsifiers that would otherwise be utilized.

Pharmaceutical agents may also be prepared as small, solid particles that are small enough to safely provide an efficacious pharmaceutical dose. Such dispersions are stabilized in much the same fashion as emulsions—by adding surface-active components to stabilize the solid-liquid interface.

We describe herein a general method of preparing dispersions in which the pharmaceutical agent possesses surface-active properties and is its own surface-active dispersant.

### SUMMARY OF THE INVENTION

The present invention provides a composition of a dispersion of an organic material (the “active agent”). The dispersion includes a multiphase system having an organic phase and an aqueous phase. The agent is surface active and acts as a dispersing agent. The agent is preferably poorly water-soluble and has surfactant properties. The agent can be an anionic surfactant, a cationic surfactant, a zwitterionic surfactant, a nonionic surfactant or a biological surface-active molecule. The dispersion may be an emulsion, suspension, or association colloid (also known as micellar dispersion).

In an embodiment, the active agent is a surface active, pharmaceutically effective organic compound that is amphiphilic, having an ionic portion, which can be cationic or anionic, and a nonionic portion. The ionic portion can be formed by protonation or deprotonation of the compound by a method such as adjusting the pH of the system.

The organic phase is preferably is a water immiscible organic material. A preferred water immiscible organic material is an oil, such as a vegetable oil. The organic phase may also be comprised of the active agent itself, or the active agent in combination with other solid or semi-solid organic materials.

In an embodiment of the present invention, the dispersion does not contain any other surface-active agents other than the active agent itself. In another embodiment, the dispersion may contain one or more surface modifiers that can be cationic, anionic, nonionic, or biological, and in which the active agent still constitutes the majority of surface-active material.

The active agent is preferably a therapeutic agent, and the composition is suitable for delivery in vivo by an administrative route such as parenteral, oral, ophthalmic, topical, buccal, rectal, vaginal, transdermal or the like.

The active agent may also be formulated in a dispersion for veterinary use. In another application, the active agent may be formulated in a dispersion for cosmetic use. In yet another application, the active agent may be formulated in a dispersion for agricultural use.

These and other aspects and attributes of the present invention will be discussed with reference to the following drawings and accompanying specification.

### DETAILED DESCRIPTION OF THE INVENTION

The present invention is susceptible of embodiments in many different forms. Preferred embodiments of the invention are disclosed with the understanding that the present disclosure is to be considered as exemplifications of the principles of the invention and are not intended to limit the broad aspects of the invention to the embodiments illustrated.

The present invention provides a dispersion, solid or liquid, of an active agent. The dispersion includes a multiphase system having an organic phase and an aqueous phase. The active agent, preferably poorly water soluble, is itself surface active and acts as a dispersant or stabilizer in the dispersion.

Multiphase System

What is meant by the term “multiphase system” is a dispersion having at least one organic phase and at least one aqueous phase. The dispersion may be an emulsion (liquid-in-liquid dispersion), a suspension (solid-in-liquid dispersion), or an association colloid (also known as micellar dispersion). In one preferred form of the invention, the dispersion is an oil-in-water (O/W) emulsion in which the water phase forms the continuous phase and the oil phase forms the dispersed phase. The organic phase is preferably a water immiscible organic compound or a mixture of two or more organic compounds. The active agent is dissolved in the organic phase. The organic phase may consist of an oil such as soybean, safflower, cannola, peanut, olive and other vegetable oils. Alternatively, the organic phase may consist one or more water-immiscible compounds such as hydrocarbons, esters, amides, ethers, ketones, amines, alcohols, and the like. In yet another embodiment, the organic phase may consist of only the active agent. The ratio by weights of the organic phase to the aqueous phase is from about 1:99 to about 99:1, more preferably from 1:99 to about 3:95, and most preferably from about 1:99 to about 5:95, or any range or combination of ranges therein. The present invention further contemplates utilizing reverse emulsions or water-in-oil emulsion (W/O) where the water-immiscible organic phase forms the continuous phase and water the dispersed phase. The present invention further contemplates utilizing emulsions having more than two phases such as an oil-in-water-in-oil emulsion (O/W/O) or a water-in-oil-in-water emulsion (W/O/W), in which the oil may be any water-immiscible organic phase that is a fluid. One embodiment of the present invention is intended in forming a liquid in liquid dispersion multiphase system.

In the instance of a liquid-in-liquid dispersion, what is meant by the term “water immiscible organic phase” are those organic compounds in their liquid state which form an interfacial meniscus when combined with an aqueous solution in quantities that exceed their aqueous solubility. In a preferred form of the invention, the water immiscible organic phase consisting of a liquid will have a vapor pressure higher than that of water when both the organic phase and water are measured at room temperature. Suitable water immiscible organic liquids include, but are not limited to, substituted or unsubstituted, linear, branched or cyclic alkanes with a carbon number of 5 or higher, substituted or unsubstituted, linear, branched or cyclic alkenes with a carbon number of 5 or higher, substituted or unsubstituted, linear, branched or cyclic alkynes with a carbon number of 5 or higher; aromatic hydrocarbons completely or partially halogenated hydrocarbons, ethers, esters, ketones, mono-, di- or tri-glycerides, native oils, alcohols, aldehydes, acids, amines, linear or cyclic silicones, hexamethyldisiloxane, or any combination of these liquids. Halogenated, liquid compounds include, but are not limited to carbon tetrachloride, methylene chloride, chloroform, tetrachloroethylene, trichloroethylene, trichloroethane, hydrofluorocarbons, chlorinated benzene (mono, di, tri), trichlorofluoromethane. Particularly suitable organic liquids are methylene chloride, chloroform, diethyl ether, toluene, xylene and ethyl acetate. A preferred water-immiscible organic phase is an oil, such as a vegetable oil from, for example, soybean, olive, cottonseed, safflower, cannola, peanut and the like.

The aqueous phase in the multiphase system is an aqueous solvent. This aqueous phase may be water by itself. This aqueous phase may also contain buffers, salts, surfactant(s), water-soluble polymers, and combinations of these excipients.

An embodiment of the invention also consists of a solid organic phase of an active agent dispersed in a continuous liquid phase. In this embodiment, the active agent acts as the principal stabilizer at the interface between the solid phase and the liquid phase.

The Active Agent Serving as its Own Dispersant, or Surface Stabilizing Agent

A “dispersant” or “surface stabilizing agent” is a compound that modifies the boundary between two phases. A surface-stabilizing agent reduces the interfacial tension between two immiscible fluids, or between a solid and fluid, solid and gas, or liquid and gas. An example of stabilization at a solid-liquid or liquid-liquid interface is represented by the organic phase and the aqueous phase in the multiphase system of the present invention. The surface-stabilizing agent, consists principally of the active agent, and can be an anionic surfactant, a cationic surfactant, a zwitterionic surfactant, a nonionic surfactant or a biological surface-active molecule.

In an embodiment, the active agent is surface active, acting as its own surface-stabilizing agent, by interacting with the continuous phase at the interface between the particle or droplet surface and the continuous phase. It can interact at this interface by being partially ionic, cationic or anionic, to provide a mixture of charged (ionic) and uncharged (nonionic) molecular species of the compound. Surface stabilization may also occur by non-ionic interactions between the particle or droplet surface and the continuous phase. In this case, the active agent, also serving as the surface stabilizing agent, interacts with the aqueous phase via hydrogen bonding or other dipolar interactions. In an oil-in-water emulsion, the uncharged fraction of the active agent would dissolve within the interior of the droplet composed of the organic phase while the charged or polar fraction interacts with the aqueous phase to stabilize the interface between the two phases. In a suspension (solid-in-liquid dispersion), the dispersed solid particles contain, whole or in part, the active agent, a portion of which is partially charged or polar. The charged or polar fraction of the active agent interacts with the aqueous phase to stabilize the solid-liquid interface. In the case that the active agent is charged, the active agent can be made partially ionic by deprotonation to form negatively charged species of the compound, by protonation to form positively charged species of the compound. The solid-in-liquid dispersion can also be formed by disposition of molecule of active agent at the phase boundary with polar functional groups at the periphery and interacting through hydrogen bonding with the aqueous continuous phase. Charge stabilization of the interface can be accomplished by adjusting the pH of the formulation so that an acid-base equilibrium would exist between charged (ionized) and uncharged (non-ionized) species of the compound. The pH of the formulation can be stabilized by adding appropriate pH adjusting agents. Examples of such agents are sodium hydroxide, hydrochloric acid, tris buffer, citrate buffer, acetate, lactate, meglumine and the like. In a preferred embodiment, the pH-adjusting agent is added to the system to bring the pH of the formulation within the range of from about 3 to about 11. Active agents with polar functional moieties, hydroxy groups for example, would diffuse to the interface and undergo molecular rearrangement at the surface so that interaction with the aqueous phase affords maximum reduction of surface free energy.

The active agent used in the present invention is preferably poorly water-soluble. What is meant by “poorly water soluble” is a solubility of the compound in water of less than about 10 mg/mL, and preferably less than 1 mg/mL. These poorly water-soluble agents are most suitable for aqueous suspension preparations since there are limited alternatives of formulating these agents in an aqueous medium.

This active agent can be selected from pharmaceutical agents such as therapeutic agents nutritional supplements, and diagnostic agents. In this invention, the active agent can also be selected from cosmetics, or from agricultural agents such as pesticides, herbicides, and the like.

Within the class of pharmaceutical agents, the therapeutic agents can be selected from a variety of known pharmaceuticals such as, but not limited to: analgesics, anesthetics, analeptics, adrenergic agents, adrenergic blocking agents, adrenolytics, adrenocorticoids, adrenomimetics, anticholinergic agents, anticholinesterases, anticonvulsants, alkylating agents, alkaloids, allosteric inhibitors, anabolic steroids, anorexiants, antacids, antidiarrheals, antidotes, antifolics, antipyretics, antirheumatic agents, psychotherapeutic agents, neural blocking agents, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antimalarials, antiseptics, antineoplastic agents, antiprotozoal agents, immunosuppressants, immunostimulants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, hemostatics, hematological agents, hemoglobin modifiers, hormones, hypnotics, immuriological agents, antihyperlipidemic and other lipid regulating agents, muscarinics, muscle relaxants, parasympathomimetics, parathyroid calcitonin, prostaglandins, radio-pharmaceuticals, sedatives, sex hormones, anti-allergic agents, stimulants, sympathomimetics, thyroid agents, vasodilators, vaccines, vitamins, and xanthines. Antineoplastic, or anticancer agents, include but are not limited to paclitaxel and derivative compounds, and other antineoplastics selected from the group consisting of alkaloids, antimetabolites, enzyme inhibitors, alkylating agents and antibiotics. The therapeutic agent can also be a biologic, which includes but is not limited to proteins, polypeptides, carbohydrates, polynucleotides, and nucleic acids. The protein can be an antibody, which can be polyclonal or monoclonal.

Within the class of pharmaceutical agents, the diagnostic agents include ionic and non-ionic X-ray contrast media, magnetic resonance imaging agents, or ultrasound imaging agents. Preferred contrast agents include those that are expected to disintegrate relatively rapidly under physiological conditions, thus minimizing any particle associated inflammatory response. Disintegration may result from enzymatic hydrolysis, solubilization of carboxylic acids at physiological pH, or other mechanisms. Thus, poorly soluble iodinated carboxylic acids such as iodipamide, diatrizoic acid, and metrizoic acid, along with hydrolytically labile iodinated species such as WIN 67721, WIN 12901, WIN 68165, and WIN 68209 or others may be preferred. Magnetic resonance imaging agents include gadopentate, and other paramagnetic metal complexes. Ultrasound imaging agents for echocontrast include microbubbles, liposomal formulations and other acoustically reflective dispersions.

A description of these classes of therapeutic agents and diagnostic agents and a listing of species within each class can be found in Martindale, The Extra Pharmacopoeia, Twenty-ninth Edition, The Pharmaceutical Press, London, 1989 which is incorporated herein by reference and made a part hereof. The therapeutic agents and diagnostic agents are commercially available and/or can be prepared by techniques known in the art.

Examples of nutritional supplements contemplated for use in the practice of the present invention include, but are not limited to, proteins, carbohydrates, water-soluble vitamins (e.g., vitamin C, B-complex vitamins, and the like), fat-soluble vitamins (e.g., vitamins A, D, E, K, and the like), and herbal extracts. The nutritional supplements are commercially available and/or can be prepared by techniques known in the art.

A cosmetic agent is any active ingredient capable of having a cosmetic activity. Examples of these active ingredients can be, inter alia, emollients, humectants, free radical-inhibiting agents, anti-inflammatories, vitamins, depigmenting agents, anti-acne agents, antiseborrhoeics, keratolytics, slimming agents, skin coloring agents and sunscreen agents, and in particular linoleic acid, retinol, retinoic acid, ascorbic acid alkyl esters, polyunsaturated fatty acids, nicotinic esters, tocopherol nicotinate, unsaponifiables of rice, soybean or shea, ceramides, hydroxy acids such as glycolic acid, selenium derivatives, antioxidants, beta-carotene, gamma-orizanol and stearyl glycerate. The cosmetics are commercially available and/or can be prepared by techniques known in the art.

In this invention the active agent also includes preparations for agricultural use. This includes pesticides, herbicides, fungicides, plant nutrients and supplements. Examples of compound classes to which the pesticide in the present invention may belong include ureas, triazines, triazoles, carbamates, phosphoric acid esters, dinitroanilines, morpholines, acylalanines, pyrethroids, benzilic acid esters, diphenylethers and polycyclic halogenated hydrocarbons. Specific examples of pesticides in each of these classes are listed in Pesticide Manual, 9th Edition, British Crop Protection Council. The pesticides are commercially available and/or can be prepared by techniques known in the art.

Droplet or Particle Sizes of the Dispersion

The size of the solid particles or droplets in the dispersion of the present invention have an average effective particle size of generally less than about 100 μm as measured by dynamic light scattering methods, e.g., photocorrelation spectroscopy, laser diffraction, low-angle laser light scattering (LALLS), medium-angle laser light scattering (MALLS), light obscuration methods (Coulter method, for example), rheology, or microscopy (light or electron). However, the particles can be prepared in a wide range of sizes, such as from about 20 μm to about 10 nm, from about 10 μm to about 10 nm, from about 2 μm to about 10 mn, from about 1 μm to about 10 nm, from about 400 mn to about 50 nm, from about 200 nm to about 50 nm or any range or combination of ranges therein. The preferred average effective particle size depends on factors such as the intended route of administration, formulation, solubility, toxicity and bioavailability of the compound.

To be suitable for pharmaceutical use, the droplets or particles fall within a broad size range, depending on route of administration, and application. For example, to be suitable for parenteral administration, the droplets or particles preferably have an average effective particle size of less than about 7 μm, and more preferably less than about 2 μm or any range or combination of ranges therein. Parenteral administration includes intravenous, intra-arterial, intrathecal, intraperitoneal, intraocular, intra-articular, intradural, intraventricular, intrapericardial, intramuscular, intradermal or subcutaneous injection. Droplet or particle sizes for oral dosage forms can be in excess of 2 μm, and range up to about 100 μm, provided that the droplets or particles have sufficient bioavailability and other characteristics of an oral dosage form.

Co-Surfactants

The dispersion of the present invention does not require the use of any other surface stabilizing agents, except in minor amounts, since the active agent is itself the major stabilizer. However, co-surfactants may be used in which the dispersion may have one or more optional surface modifiers such as an anionic surfactant, a cationic surfactant, a nonionic surfactant or a biologically surface active molecule added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.). Suitable cationic surfactants include but are not limited to quaternary ammonium compounds, such as benzalkonium chloride, cetyltrimethylammonium bromide, chitosans, lauryldimethylbenzylammonium chloride, acyl camitine hydrochlorides, or alkyl pyridinium halides. As anionic surfactants, phospholipids may be used. Suitable phospholipids include, for example phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)), phosphatidylserine, phosphatidylinositol, phosphatidylglycerol, phosphatidic acid, lysophospholipids, egg or soybean phospholipid or a combination thereof. The phospholipid may be salted or desalted, hydrogenated or partially hydrogenated or natural semisynthetic or synthetic. The phospholipid may also be conjugated with a water-soluble or hydrophilic polymer. A preferred polymer is polyethylene glycol (PEG), which is also known as the monomethoxy polyethyleneglycol (mPEG). The molecule weights of the PEG can vary, for example, from 200 to 50,000. Some commonly used PEG's that are commercially available include PEG 350, PEG 550, PEG 750, PEG 1000, PEG 2000, PEG 3000, and PEG 5000. The phospholipid or the PEG-phospholipid conjugate may also incorporate a functional group that can covalently attach to a ligand including but not limited to proteins, peptides, carbohydrates, glycoproteins, antibodies, or pharmaceutically active agents. These functional groups may conjugate with the ligands through, for example, amide bond formation, disulfide or thioether formation, or biotin/streptavidin binding. Examples of the ligand-binding functional groups include but are not limited to hexanoylamine, dodecanylamine, 1,12-dodecanedicarboxylate, thioethanol, 4-(p-maleimidophenyl)butyramide (MPB), 4-(p-maleimidomethyl)cyclohexane-carboxamide (MCC), 3-(2-pyridyldithio)propionate (PDP), succinate, glutarate, dodecanoate, and biotin.

Suitable nonionic surfactants include: polyoxyethylene fatty alcohol ethers (Macrogol and Brij), polyoxyethylene sorbitan fatty acid esters (Polysorbates), polyoxyethylene fatty acid esters (Myij), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethylcellulose, hydroxypropylcellulose, hydroxypropylmethylcellulose, noncrystalline cellulose, polysaccharides including starch and starch derivatives such as hydroxyethylstarch (HES), polyvinyl alcohol, and polyvinylpyrrolidone. In a preferred form of the invention, the nonionic surfactant is a polyoxyethylene and polyoxypropylene copolymer and preferably a block copolymer of propylene glycol and ethylene glycol. Such polymers are sold under the tradename POLOXAMER also sometimes referred to as PLURONIC®, and sold by several suppliers including Spectrum Chemical and Ruger. Among polyoxyethylene fatty acid esters is included those having short alkyl chains. One example of such a surfactant is SOLUTOL® HS 15, polyethylene-660-hydroxystearate, manufactured by BASF Aktiengesellschaft.

Surface-active biological molecules include such molecules as albumin, casein, hirudin or other appropriate proteins. Polysaccharide biologics are also included, and consist of but not limited to, starches, heparin and chitosans.

The surface modifiers are commercially available and/or can be prepared by techniques known in the art. Two or more surface modifiers can be used in combination.

In a preferred embodiment, the active agent still constitutes the majority of the surface-active material in the embodiment in which a co-surfactant is included in the dispersion. For example, the co-surfactant is present in less than 50% by weight of the active agent.

The active agent can also be used in combination with other active agents. For example, the active agent, acting as a dispersant, can be used to coat a solid drug nanoparticle (similar to a non-therapeutic surfactant). Examples include nanoparticles of paclitaxel coated with C-6 ceramide or coating the nanoparticle with other agents. Another example of active agent combinations is with phospholipids. Phospholipids can be used to coat solid drug nanoparticles (for stabilization) and additional active agents (e.g., tetracaine, lidocaine, benzocaine, dibucaine, etidocaine, etc.) can be dispersed within the bilayer of such phospholipid coating in order to improve stability the phopholipid bilayer and/or provide additional therapeutic benefit.

Pharmaceutical Dosage Forms and Formulations

In a preferred embodiment of the present invention, the active agent is a therapeutically useful and the composition is suitable for use as a pharmaceutical composition. In an embodiment, the composition is sterile. Methods to sterilize the composition are well known in the art, including but are not limited to, sterile filtration, heat sterilization, high-pressure sterilization, and gamma irradiation. In another embodiment, the composition further includes an osmolality adjusting agent, such as, but not limited to, glycerin and trehalose.

One preferred route of delivery of the composition is by parenteral route. To be suitable for parenteral administration, the droplets preferably have an average effective particle size of less than about 7 μm, and more preferably less than about 2 μm or any range or combination of ranges therein. Parenteral administration includes intravenous, intra-arterial, intrathecal, intraperitoneal, intraocular, intra-articular, intradural, intraventricular, intrapericardial, intramuscular, intradermal or subcutaneous injection.

Another preferred route is the oral route. Oral dosage forms include capsules, caplets, soft and hard gel capsules, or other delivery vehicle for delivering a drug by oral administration. Droplet or particle sizes for oral dosage forms can be in excess of 2 μm, and can range in size up to about 100 μm, provided that the droplets or particles have sufficient bioavailability and other characteristics of an oral dosage form.

Dosage forms for other routes of delivery, such as topical, ophthalmic, buccal, rectal, vaginal, transdermal and the like can also be formulated from the dispersions made from the present invention.

Methods for Preparing Self-stabilizing Dispersions

Methods to prepare dispersed systems are well documented and are well known in the art. For example, a typical method of preparing an emulsion of a poorly water soluble compound includes the steps of: (1) dissolving the compound in a water immiscible organic phase; and (2) emulsifying the organic phase with an aqueous phase in the presence of an emulsifying agent to form a multiphase phase system in which oil droplets of the organic compound are suspended in the continuous aqueous phase. The emulsifier stabilizes the interface between the organic phase and the aqueous phase to form stable droplets of the organic compound. The droplets can be further reduced to a desired size by applying high shear mixing (e.g., homogenization). In the present invention, the organic compound possesses surface-active properties and itself serves as its own dispersant or surface-stabilizing agent (emulsifier) so that additional dispersants or emulsifying agents are not required. Although additional co-surfactants can be used in the present invention, the level of the co-surfactants required can be substantially reduced as compared to conventional emulsions, and the majority of surface stabilizing agent consists of the active agent.

Examples of methods for preparing emulsions of the present invention for poorly water soluble organic compounds are disclosed in co-pending and commonly assigned U.S. patent application Ser. Nos. 09/964,273 and 10/183,035, which are incorporated herein by reference and made a part hereof.

### EXAMPLE 1

### Efaproxiral as a Potential Surface Active, Poorly Water-Soluble Active Agent

Efaproxiral (chemical name 2-(4-2-((3,5-dimethylphenyl)amino)-2-oxoethyl)phenoxy)-2-methylproprionic acid, also known as RSR13, under development by Allos Therapeutics) is an organic acid that can be deprotonated to form a molecular species with anionic surfactant properties.

### EXAMPLE 2

### Prostaglandins as Potential Surface-Active, Poorly Water-Soluble Active Agents

Prostaglandins (e.g., prostaglandin E₁, also known as alprostadil) are carboxylic acids that may be deprotonated to form an amphipathic salt that is potentially capable of stabilizing an oil-in-water or solid-water interface.

### EXAMPLE 3

### Amiodarone as a Potential Surface Active, Poorly Water-Soluble Active Agent

Amiodarone is a highly lipophilic drug (log P=6.99) that is also an amine. At low pH, protonation of the amino group affords a molecule that is positively charged and can act as a cationic surfactant. This property may be used to form an emulsion in which the protonated drug stabilizes the surface of oil droplets that comprise an emulsion. In this case, as well as previously described molecules of anionic, surface active drugs, a significant fraction of the drug might also be dissolved as a non-ionized form within the interior of the oil droplet. By adjusting the pH to provide enough ionized drug to coat every oil droplet, electrostatic stabilization of the emulsion droplets can be assured.

### EXAMPLE 4

### Betulinic Acid as a Potential Surface Active, Poorly Water-Soluble Active Agent, and Potential Formulations

Betulinic acid is a triterpene that is present in many plant tissues and is one of the most abundant plant-based compounds, the saponins. It can also be synthesized from betulin, a substance that is found in the bark of the white birch. Some studies have indicated that it can selectively induce apoptosis in melanoma cells and may be of some benefit in the treatment of this type of cancer. It may also have potential in HIV treatment, as it appears to inhibit HIV replication through inhibition of viral fusion to T cells.

We have predicted the pKa of betulinic acid to be around 4.9 (SPARC online pKa estimation program, http://ibmlc2.chem.uga.edu/sparc/). Below pH 4, drug solubility should be minimal. The predicted octanol-water partition coefficient (Log K_{OW}) is 6.86 (HyperChem, release 5.11 Pro, 1999, Hypercube, Inc.). It is conceivable to use the drug itself as a surface stabilizer because a significant fraction will be negatively charged near or above the pKa of 4.9. Because of the high predicted Log K_{OW} value, the anionic form should be strongly amphiphilic. The percentage of total drug that would be ionized at each pH, based on the calculated pKa, is as follows: pH 4 (11%), pH 4.5 (28%), pH 5 (56%).

The acidity of betulinic acid and its expected amphipathic behavior suggests four reasonable formulation paths. In the first approach, the drug itself is used as its own surface-stabilizing agent by formulating near or above the drug pKa. In a second approach, the drug is deliberately formulated at high pH (8) with phospholipids and one or more ionic co-surfactants with the expectation that it form ternary mixed micelles. In yet another option, a mixed-micelle dispersion may be possible by formulating at high pH, above the drug pKa, and with only phospholipids to act as a co-surfactant. In this case the drug acts as its own anionic surfactant, with behavior similar to that of a bile salt, interacting with the phospholipids to form a binary mixed surfactant system, and potentially forming mixed micelles. These options are presented in Table 1.

TABLE 1
Formulation Options for Betulinic Acid

| Option | Type | pH Range | Phospholipids | Bile salt |
|---|---|---|---|---|
| 1 | Nanosuspension | >4 | No | None* |
| 2 | Mixed micelle or | 7-8.5 | Yes | Yes (forms |
| | nanoparticle | | | ternary mixed |
| | | | | phase) |
| 3 | Mixed micelle or | 7-8.5 | Yes | None** |
| | nanoparticle | | | |

*In this case, the drug may serve as is own surfactant.
**The phospholipids may form a binary mixed phase with ionized drug.

Different surfactant packages and proposed test ranges are shown in Table 2. This table is intended as a starting point, and other surfactants may be also screened, depending on the results obtained from the initial list. All excipients will be those recognized for pharmaceutical application, and known to be tolerated upon intravenous administration. Phospholipids (e.g., phosphatidyl choline) that we propose using are currently used in I.V. emulsions (for example, IVELIP and INTRALIPID). Poloxamer 188 is currently used in a number of pharmaceutical products.

Hetastarch (hydroxyethyl starch) is a constituent of HESPAN, which is owned and marketed by Braun AG.

Osmolality may be adjusted with either glycerin or trehalose.

TABLE 2
List of potential excipients (osmolality adjusted with glycerol or trehalose)

| | | Concentration |
|---|---|---|
| | Surfactants | Range (%, w/v) |
| A | Phospholipids (e.g., phosphatidyl choline, DMPG) | 1 to 5 |
| B | Poloxamer 188 (F68) | 0.1 to 0.5 |
| C | Sodium deoxycholate | 0.05 to 0.5 |
| D | Sodium taurocholate | 0.05 to 0.5 |
| E | Sodium glycocholate | 0.05 to 0.5 |
| F | Hetastarch (500,000-700,000 MW) | 0.5 to 3 |
| G | Solutol^{a} | 0.05 to 0.5 |
| H | pegylated phospholipids (e.g., mPEG-DMPG)^{b} | 0.05 to 0.5 |

^{a}SOLUTOL is the brand name for PEG-600 12-hydroxystearate from BASF AG.
^{b}Medium-length polyethylene glycol-dimyristoylphosphatidylglycerol

While specific embodiments have been illustrated and described, numerous modifications come to mind without departing from the spirit of the invention and the scope of protection is only limited by the scope of the accompanying claims.

1. A composition of a dispersion of an active agent comprising a multiphase system having an organic phase and an aqueous phase, wherein the active agent has surfactant properties and acts as a surface-stabilizing agent for the dispersion.

2. The composition of claim 1, wherein the active agent is an anionic surfactant, a cationic surfactant, a zwitterionic surfactant, a nonionic surfactant or a biological surface active molecule.

3. The composition of claim 1, wherein the active agent is amphiphilic having an ionic portion and a non-ionic portion.

4. The composition of claim 3, wherein the ionic portion is cationic, anionic, or zwitterionic.

5. The composition of claim 4, wherein the ionic portion is formed by protonation or deprotonation.

6. The composition of claim 5, wherein the protonation or deprotonation is the result of adjusting the pH of the system.

7. The composition of claim 1, wherein the dispersion is a liquid-in-liquid dispersion.

8. The composition of claim 7, wherein the dispersion is an oil-in-water (O/W) emulsion.

9. The composition of claim 7, wherein the dispersion is a water-in-oil (W/O) emulsion.

10. The composition of claim 7, wherein the dispersion is a water-in-oil-in-water (W/O/W) emulsion.

11. The composition of claim 7, wherein the dispersion is an oil-in-water-in-oil (O/W/O) emulsion.

12. The composition of claim 1, wherein the dispersion is a solid-in-liquid dispersion.

13. The composition of claim 1, wherein the dispersion is a micellar dispersion.

14. The composition of claim 1, wherein the dispersion does not contain any other dispersant or emulsifying agent.

15. The composition of claim 1, wherein the dispersion further comprising one or more surface modifiers selected from the group consisting of: anionic surfactants, cationic surfactants, zwitterionic surfactants, nonionic surfactants and surface active biological modifiers.

16. The composition of claim 15, wherein the anionic surfactant is selected from the group consisting of: alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, bile acids and their salts, cholic acid, deoxycholic acid, glycocholic acid, taurocholic acid, and glycodeoxycholic acid.

17. The composition of claim 15, wherein the cationic surfactant is selected from the group consisting of quaternary ammonium compounds, benzalkonium chloride, cetyltrimethylammonium bromide, chitosans, lauryldimethylbenzylammonium chloride, acyl carnitine hydrochlorides and alky pyridinium halides.

18. The composition of claim 15, wherein the nonionic surfactant is selected from the group consisting of: polyoxyethylene fatty alcohol ethers, polyoxyethylene sorbitan fatty acid esters, polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers, poloxamines, methylcellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, and polyvinylpyrrolidone.

19. The composition of claim 15, wherein the surface active biological modifiers are selected from the group consisting of: albumin, casein, hirudin, or other proteins.

20. The composition of claim 15, wherein the surface active biological modifiers are polysaccharides.

21. The composition of claim 20, wherein the polysaccharide is selected from the group consisting of a starch, heparin, chitosan.

22. The composition of claim 15, wherein the surface modifier comprises a phospholipid selected from natural phospholipids and synthetic phospholipids.

23. The composition of claim 22, wherein the phospholipid is selected from the group consisting of: phosphatidylcholine, phosphatidylethanolamine, diacyl-glycero-phosphoethanolamine, dimyristoyl-glycero-phosphoethanolamine (DMPE), dipalmitoyl-glycero-phosphoethanolamine (DPPE), distearoyl-glycero-phosphoethanolamine (DSPE), dioleolyl-glycero-phosphoethanolamine (DOPE), phosphatidylserine, phosphatidylinositol, phosphatidylglycerol, phosphatidic acid, lysophospholipids, polyethylene glycol-phospholipid conjugates, egg phospholipid and soybean phospholipid.

24. The composition of claim 22, wherein the phospholipid further comprises a functional group to covalently link to a ligand.

25. The composition of claim 24, wherein the ligand is selected from the group consisting of proteins, peptides, carbohydrates, glycoproteins, antibodies and pharmaceutically active agents.

26. The composition of claim 24, wherein the functional group is selected from the group consisting of: hexanoylamine, dodecanylamine, 1,12-dodecanedicarboxylate, thioethanol, 4-(p-maleimidophenyl)butyramide (MPB), 4-(p-maleimidomethyl)cyclohexane-carboxamide (MCC), 3-(2-pyridyldithio)propionate (PDP), succinate, glutarate, dodecanoate, and biotin.

27. The composition of claim 15, wherein the surface modifier comprises a bile acid or a salt thereof.

28. The composition of claim 27, wherein the surface modifier is selected from deoxycholic acid, glycocholic acid, glycodeoxycholic acid, taurocholic acid and salts of these acids.

29. The composition of claim 15, wherein the surface modifier comprises a copolymer of oxyethylene and oxypropylene.

30. The composition of claim 1, wherein the organic phase comprises a solid drug nanoparticle coated with an additional active agent surfactant.

31. The composition of claim 15, wherein the surface modifier is less than 50% by weight of the active agent.

32. The composition of claim 1, wherein the organic phase comprises a water immiscible solvent.

33. The composition of claim 32 wherein the water immiscible solvent is selected from the group consisting of: linear, branched or cyclic alkanes with carbon number of 5 or higher, linear, branched or cyclic alkenes with carbon number of 5 or higher, linear, branched or cyclic alkynes with carbon number of 5 or higher; aromatic hydrocarbons completely or partially halogenated hydrocarbons, ethers, esters, ketones, mono-, di- or tri-glycerides, native oils, alcohols, aldehydes, acids, amines, linear or cyclic silicones, hexamethyldisiloxane, or any combination of these solvent.

34. The composition of claim 32, wherein the water immiscible solvent is an oil.

35. The composition of claim 34, wherein the oil is a vegetable oil.

36. The composition of claim 35, wherein the vegetable oil is selected from the group consisting of: soybean, olive, cottonseed, safflower, cannola, and peanut.

37. The composition of claim 32, wherein the water immiscible solvent has a vapor pressure higher than water at room temperature.

38. The composition of claim 1, wherein the organic phase comprises a partially water miscible solvent.

39. The composition of claim 38, wherein the partially water miscible solvent is selected from the group consisting of: fluorinated solvents, tetrahydrofuran, propylene carbonate, benzyl alcohol, and ethyl acetate.

40. The composition of claim 32, wherein the organic phase further includes a co-solvent.

41. The process of claim 40, wherein the co-solvent is a water miscible organic solvent.

42. The composition of claim 1 further comprising a pH adjusting agent.

43. The composition of claim 42, wherein the pH adjusting agent is selected from the group consisting of sodium hydroxide, hydrochloric acid, tris buffer, citrate buffer, acetate, lactate, and meglumine.

44. The composition of claim 42, wherein the pH adjusting agent is added to the system to bring the pH of the aqueous phase within the range of from about 3 to about 11.

45. The composition of claim 1 further comprising an osmolality adjusting agent.

46. The composition of claim 45, wherein the osmolality adjusting agent is selected from the group consisting of glycerin and trehalose.

47. The composition of claim 1, wherein the organic phase is a solid organic material.

48. The composition of claim 1, wherein the multiphase has a ratio of the organic phase to the aqueous phase of from about 1:99 to about 99:1

49. The composition of claim 1, wherein the multiphase has a ratio of the organic phase to the aqueous phase greater than about 3:97.

50. The composition of claim 1, wherein the multiphase has a ratio of the organic phase to the aqueous phase greater than about 5:95.

51. The composition of claim 1, wherein the active agent is poorly water soluble.

52. The composition of claim 51, wherein the active agent has a solubility in water of less than about 10 mg/mL.

53. The composition of claim 51, wherein the active agent has a solubility in water of less than about 1 mg/mL.

54. The composition of claim 1, wherein the active agent is selected from the group consisting of therapeutic agents, diagnostic agents, cosmetics, nutritional supplements, and pesticides.

55. The composition of claim 54, wherein the therapeutic agent is selected from the group consisting of analgesics, anesthetics, analeptics, adrenergic agents, adrenergic blocking agents, adrenolytics, adrenocorticoids, adrenomimetics, anticholinergic agents, anticholinesterases, anticonvulsants, alkylating agents, alkaloids, allosteric inhibitors, anabolic steroids, anorexiants, antacids, antidiarrheals, antidotes, antifolics, antipyretics, antirheumatic agents, psychotherapeutic agents, neural blocking agents, anti-inflammatory agents, antihelmintics, anti-arrhythmic agents, antibiotics, anticoagulants, antidepressants, antidiabetic agents, antiepileptics, antifungals, antihistamines, antihypertensive agents, antimuscarinic agents, antimycobacterial agents, antimalarials, antiseptics, antineoplastic agents, antiprotozoal agents, immunosuppressants, immunostimulants, antithyroid agents, antiviral agents, anxiolytic sedatives, astringents, beta-adrenoceptor blocking agents, contrast media, corticosteroids, cough suppressants, diagnostic agents, diagnostic imaging agents, diuretics, dopaminergics, hemostatics, hematological agents, hemoglobin modifiers, hormones, hypnotics, immuriological agents, antihyperlipidemic and other lipid regulating agents, muscarinics, muscle relaxants, parasympathomimetics, parathyroid calcitonin, prostaglandins, radio-pharmaceuticals, sedatives, sex hormones, anti-allergic agents, stimulants, sympathomimetics, thyroid agents, vasodilators, vaccines, vitamins, and xanthines.

56. The composition of claim 54, wherein the therapeutic agent is selected from the group consisting of efaporxiral, a prostaglandin, amiodarone and betulinic acid.

57. The composition of claim 1, wherein the dispersion has an average effective particle or droplet size of from about 20 μm to about 10 nm.

58. The composition of claim 1, wherein the dispersion has an average effective particle or droplet size of from about 2 μm to about 10 nm.

59. The composition of claim 1, wherein the dispersion has an average effective particle or droplet size of from about 200 nm to about 50 nm.

60. The composition of claim 1, wherein the composition is sterile.

61. The composition of claim 60, wherein the composition is sterilized by sterile filtering the emulsion, heat sterilization, gamma irradiation or high-pressure sterilization.

62. The composition of claim 1 suitable for administering to a subject in need of the agent.

63. The composition of claim 62, wherein the composition is administered by a route selected from the group consisting of: parenteral, oral, ophthalmic, topical, buccal, rectal, vaginal, and transdermal.

64. The composition of claim 62, wherein the composition is administered parenterally.
